SEQUENCE LISTING (18 sequences)

SEQ ID NO: 1 - DNA, 25 bp, Artificial Sequence
  Synthetic construct
ttaacgagctgtatgtcgtgagaat

SEQ ID NO: 2 - DNA, 24 bp, Artificial Sequence
  Synthetic construct
aacgagctgtatgtcgtgagaatc

SEQ ID NO: 3 - DNA, 22 bp, Artificial Sequence
  Synthetic construct
ccctccaaatgaaattccaaca

SEQ ID NO: 4 - DNA, 20 bp, Artificial Sequence
  Synthetic construct
ggctttgttgcaaggctatg

SEQ ID NO: 5 - DNA, 22 bp, Artificial Sequence
  Synthetic construct
cgagctgtatgtcgtgagaatc

SEQ ID NO: 6 - DNA, 20 bp, Artificial Sequence
  Synthetic construct
caagcctattcagagcatgg

SEQ ID NO: 7 - DNA, 27 bp, Artificial Sequence
  Synthetic construct
atggatcatcaagctctaagaaagaac

SEQ ID NO: 8 - DNA, 25 bp, Artificial Sequence
  Synthetic construct
agtgtcgtctgtatggatcatcaag

SEQ ID NO: 9 - DNA, 21 bp, Artificial Sequence
  Synthetic construct
cctcaagcgaagatgcaaaat

SEQ ID NO: 10 - DNA, 26 bp, Artificial Sequence
  Synthetic construct
tggatcatcaagctctaagaaagaac

SEQ ID NO: 11 - DNA, 20 bp, Artificial Sequence
  Synthetic construct
gattgcaactgctcatcagg

SEQ ID NO: 12 - DNA, 19 bp, Artificial Sequence
  misc_feature (9)..(12): ribonucleotide
ataggcttgaagcagtgca

SEQ ID NO: 13 - DNA, 20 bp, Artificial Sequence
  misc_feature (9)..(12): ribonucleotide
ataggcttgaagcagtgcat

SEQ ID NO: 14 - DNA, 22 bp, Artificial Sequence
  misc_feature (11)..(14): ribonucleotide
tcagagcatggaaataaaactt

SEQ ID NO: 15 - DNA, 1720 bp, E. coli O157H7
  misc_feature (1)..(1720): I fragment of E. coli O157H7
cggaaatattgacatgggatgatgaacaatgggaggtatttgtccatgattggcttattgtctgtaaatcagatgattacccgtggagcgaacgtttgggaggagctggagataaaggtagagacgttgttggatataaatcggatcctaacgtagaaggttattcttgggataattatcaatgcaaactgtacaaaaaaagtttagggttctctgatgttgtagttgagtttggaaaacttatctattttactctgaatggtgattatcccatccctcagaagtacttttttgtggcaccctatgatttatctactacattttctaatttattgaaaaataaaaacgagcttaaaaaagcagtccttgattcatgggattcagcaatttcaaaaaaataactaaaaagattgatattccattagatgatgaaataaaaaaatatattgaggattttgattttagtattttttactctctacccttatcattgattttaaatgatattgcaaatacacacctttattttaagtactttaacgagctgtatgtcgtgagaatccctccaaatgaaattccaacatacaattcaaaaaaagagtctgtatatgttaatgcactgcttcaagcctattcagagcatggaaataaaacttatagttctttcttagagcttgatgatccatacagacgacactttaataatagtagaaatgatttttattttgcatcttcgcttgaggtttttgtccgcgaagtatttaaagatgatgtattcaaagcattgaaatgttacatttcatcttcaattgaacccgtcttttatgaagaccataattatgcatttattaggtgtaatgcagtcttgaagcaggctgttctgacaccaattgcacattcagtactatcaaaaatatgtgaagcaaatgataaaaaaggaatatgccatcatttggttaatgatggtgaagtaatttggacggtgagataatggttagaatttataattcaagtttagaagtggcatgtcgaatggcgaaagtgctcgtcgctatttatccttcttcattaagccttgaacggcttatttgttttgattttattttagtaaatcttaaggattttttacctgaagagattagtcttcatcctccaataccccgtagagatgctcagttagccctaaaacgagagattgttttagaatcattggctttgttgcaaggctatgaactagcctcaaaaatttatacacatcgtggttttgtatataaagcttctgaaaaaacatatgcatttacaaattctctacataatgaatatgttgcgcagatggagcataatataaatttggtggttaagttatatagtgatattcctgatgagcagttgcaatcaattataaaaaataaaattggcaaatatgatatggaatttaattatgaatgacaatttttttacgttcagaaaaataaaggtaaccggattcaataaattagatgctataattgaatttggttctaaattgactattttatatggtggttctgactctggaaaaacatacatatattatttgattcgatatttattagggagtgaaaaactaaaaaataaagatatcgatcatgctcaaggttatgatttagcctatctggaatttaattttcaaggtagggtaatgacaattgagaggtctcttcaggatagcgcccattac

SEQ ID NO: 16 - DNA, 26 bp, Artificial Sequence
  misc_feature (1)..(2): absence or t
nnnncgagctgtatgtcgtgagaatn

SEQ ID NO: 17 - DNA, 20 bp, Artificial Sequence
  misc_feature (20)..(20): absence or t
ataggcttgaagcagtgcan

SEQ ID NO: 18 - DNA, 22 bp, Artificial Sequence
  misc_feature (10)..(10): deoxyribonucleotide or ribonucleotide
tcagagcatggaaataaaactt

(19) United States
(12) Patent Application Publication
(10) Pub. No.: US 2012/0052494 A1
(43) Pub. Date: Mar. 1, 2012

(54) OLIGONUCLEOTIDES FOR DETECTING E. coli O157:H7 STRAINS AND USE THEREOF

(75) Inventors: Jun LI, Baltimore, MD (US); Win Den CHEUNG, Olney, MD (US); Jason OPDYKE, Silver Spring, MD (US)

(73) Assignee: SAMSUNG TECHWIN CO., LTD., Changwon-city (KR)

(21) Appl. No.: 13/109,043

(22) Filed: May 17, 2011

Related U.S. Application Data
(60) Provisional application No. 61/378,071, filed on Aug. 30, 2010.

(51) Int. Cl. C12Q 1/68

(52) U.S. Cl. 435/6.11; 435/6.12

(57) ABSTRACT

Oligonucleotides, a kit, and a method for detecting E. coli O157:H7 strains are provided. According to the kit for detecting E. coli O157:H7 strains and the method of detecting E. coli O157:H7 strains by using the kit, the results of the detection can be rapidly identified with a reduced number of copies of a sample in real-time.

### CROSS-REFERENCE TO RELATED APPLICATIONS

This application claims benefits from U.S. Provisional Patent Application No. 61/378,071, filed on Aug. 30, 2010, the content of which is hereby incorporated by reference in its entirety.

### FIELD

The description relates to oligonucleotides suitable for detecting E. coli O157:H7 strains as well as a kit and a method of detecting E. coli O157:H7 strains by using the oligonucleotides.

### RELATED ART

Since its first recognition in 1982 as the cause of outbreak of hemorrhagic colitis, E. coli O157:H7 was identified as one of the most widespread pathogens causing food-borne diseases in the world. E. coli O157:H7 causes thousands of illnesses in Japan and over 20,000 illnesses and over 250 deaths in the United States annually. In addition, E. coli O157:H7 strains are known as predominant pathogens of hemorrhagic colitis with Campylobacter strains, Salmonella strains, and Shigella strains. Since transmission of E. coli O157:H7 strains often occurs via food such as meat, dairy products, and drinking water, there is a need to develop a method of rapidly and economically detecting E. coli O157:H7 strains in those samples. E. coli O157:H7 strains are generally detected by culturing a sample in a selective medium, isolating strains considered as E. coli O157:H7, and identifying the strains using a biochemical or immunological method. An immunological method using an antibody provides a greater accuracy. However, an immunological method requires a large amount of a sample and production of an antibody for diagnosis.

### SUMMARY

In an embodiment, there is provided oligonucleotides suitable for a rapid, sensitive, and accurate detection of E. coli O157:H7 strains. The oligonucleotides may be a first primer including the sequence of SEQ ID NOS: 16, 3, 4, or 6; and a second primer including the sequence of SEQ ID NO: 7, 8, 9, 10, or 11. The oligonucleotides may include the sequence of SEQ ID NO: 17 or 18. In an embodiment, the first primer may have the sequence of SEQ ID NOS: 1, 2, 3, 4, 5, or 6. In an embodiment, the probe may have the sequence of SEQ ID NOS: 12, 13, or 14.

A composition including oligonucleotides suitable for a rapid, sensitive, and accurate detection of E. coli O157:H7 strains is also provided. The composition includes a first primer including the sequence of SEQ ID NOS: 16, 3, 4, or 6; and a second primer may include the sequence of SEQ ID NO: 7, 8, 9, 10, or 11. The composition may further include a probe of SEQ ID NO: 17 or 18. In an embodiment, the first primer may have the sequence of SEQ ID NOS: 1, 2, 3, 4, 5, or 6. In an embodiment, the probe may have the sequence of SEQ ID NOS: 12, 13, or 14.

In an embodiment, a kit for detecting E. coli O157:H7 strains is provided.

According to an embodiment, the kit for detecting E. coli O157:H7 strains may include a first primer including the sequence of SEQ ID NOS: 16, 3, 4, or 6, and a second primer including the sequence of SEQ ID NOS: 7, 8, 9, 10, or 11. The kit may further include a probe which is comprised of a DNA sequence and an RNA sequence. The probe may have the sequence of SEQ ID NOS: 17 or 18. In an embodiment, the first primer may be one of SEQ ID NOS: 1, 2, 3, 4, 5, or 6. In an embodiment, the probe may be one of SEQ ID NOS: 12, 13, or 14.

Various combinations of a first primer, a second primer, and a probe may be used to detect a target nucleic acid or its fragment of E. coli O157:H7. Combinations may include, but are not limited to the following examples.

A first primer including the nucleotide sequence of SEQ ID NO: 1, a second primer including the nucleotide sequence of SEQ ID NO: 7 and a probe having any one of the nucleotide sequences of SEQ ID NOS: 12 to 14;

A first primer including the nucleotide sequence of SEQ ID NO: 1, a second primer including the nucleotide sequence of SEQ ID NO: 8, and a probe having any one of the nucleotide sequences of SEQ ID NOS: 12 to 14;

A first primer including the nucleotide sequence of SEQ ID NO: 1, a second primer containing the nucleotide sequence of SEQ ID NO: 10, and a probe having any one of the nucleotide sequences of SEQ ID NOS: 12 to 14;

A first primer including the nucleotide sequence of SEQ ID NO: 2, a second primer including the nucleotide sequence of SEQ ID NO: 7, and a probe having any one of the nucleotide sequences of SEQ ID NOS: 12 to 14;

A first including the nucleotide sequence of SEQ ID NO: 2, a second primer including the nucleotide sequence of SEQ ID NO: 10 and a probe having any one of the nucleotide sequences of SEQ ID NOS: 12 to 14;

A first primer including the nucleotide sequence of SEQ ID NO: 3, a second primer including the nucleotide sequence of SEQ ID NO: 7, and a probe having any one of the nucleotide sequences of SEQ ID NOS: 12 to 14;

A first primer including the nucleotide sequence of SEQ ID NO: 3, a second primer including the nucleotide sequence of SEQ ID NO: 10, and a probe having any one of the nucleotide sequences of SEQ ID NOS: 12 to 14;

A first primer including the nucleotide sequence of SEQ ID NO: 4, a second primer including the nucleotide sequence of SEQ ID NO: 11, and a probe having any one of the nucleotide sequences of SEQ ID NOS: 12 to 14;

A first primer including the nucleotide sequence of SEQ ID NO: 5, a second primer including the nucleotide sequence of SEQ ID NO: 7, and a probe having any one of the nucleotide sequences of SEQ ID NOS: 12 to 14;

A first primer including the nucleotide sequence of SEQ ID NO: 5, a second primer including the nucleotide sequence of SEQ ID NO: 10, and a probe having any one of the nucleotide sequences of SEQ ID NOS: 12 to 14; or

A first primer including the nucleotide sequence of SEQ ID NO: 6, a second primer including the nucleotide sequence of SEQ ID NO: 9, and a probe having any one of the nucleotide sequences of SEQ ID NOS: 12 to 14.

In an embodiment, a kit for detecting E. coli O157:H7 strains may include one of the following oligonucleotides:

a primer set comprising a first primer comprising at least 10 or 15 consecutive nucleotides selected from the nucleotide sequence of SEQ ID NO: 1 and a second primer comprising at least 10 or 15 consecutive nucleotides selected from the nucleotide sequence of SEQ ID NO: 7 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 1 and a primer comprising at least 10 or 15 consecutive nucleotides selected from the nucleotide sequence of SEQ ID NO: 8 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 1 and a primer comprising at least 10 or 15 consecutive nucleotides selected from the nucleotide sequence of SEQ ID NO: 10 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 2 and a primer comprising at least 10 or 15 consecutive nucleotides selected from the nucleotide sequence of SEQ ID NO: 7 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 2 and a primer comprising at least 10 or 15 consecutive nucleotides selected from the nucleotide sequence of SEQ ID NO: 10 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 3 and a primer comprising at least 10 or 15 consecutive nucleotides selected from the nucleotide sequence of SEQ ID NO: 7 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 3 and a primer comprising at least 10 or 15 consecutive nucleotides selected from the nucleotide sequence of SEQ ID NO: 10 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 4 and a primer comprising at least 10 or 15 consecutive nucleotides selected from the nucleotide sequence of SEQ ID NO: 11 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 5 and a primer comprising at least 10 or 15 consecutive nucleotides selected from the nucleotide sequence of SEQ ID NO: 7 and a probe having any one of the nucleotide sequences of SEQ ID NOS: 12 to 14;

a primer set comprising a primer comprising at least 10 or 15 consecutive nucleotides selected from the nucleotide sequence of SEQ ID NO: 5 and a primer comprising at least 10 or 15 consecutive nucleotides selected from the nucleotide sequence of SEQ ID NO: 10 and a probe having any one of the nucleotide sequences of SEQ ID NOS: 12 to 14; or

a primer set comprising a primer comprising at least 10 or 15 consecutive nucleotides selected from the nucleotide sequence of SEQ ID NO: 6 and a primer comprising at least 10 or 15 consecutive nucleotides selected from the nucleotide sequence of SEQ ID NO: 9 and a probe having any one of the nucleotide sequences of SEQ ID NOS: 12 to 14.

In another embodiment, there is also provided a method of detecting E. coli O157:H7 strains from a sample.

The method includes (a) amplifying a target nucleic acid of E. coli O157:H7 strains in the sample to produce an increased number of copies of the target nucleic acid, the amplification including hybridizing a first primer including the sequence of SEQ ID NO: 16, 3, 4, or 6, and a second primer including the sequence of SEQ ID NO: 7, 8, 9, 10, or 11 to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the target nucleic acid to at least one probe oligonucleotide which is capable of being hybridized to the target nucleic acid to obtain a hybridized product of the target nucleic acid:probe oligonucleotide, said probe comprising a DNA sequence and an RNA sequence, and being coupled to a detectable marker; (c) contacting the hybridized product of the target nucleic acid:probe with an RNase H to cleave the probe, resulting in probe fragment dissociation from the target nucleic acid; and (d) detecting the detectable marker. The first primer may have the sequence of SEQ ID NO: 1, 2, 3, 4, 5, or 6. The probe oligonucleotide may have the oligonucleotide of SEQ ID NOs: 17 or 18. The probe oligonucleotide may be one of oligonucleotides of SEQ ID NOs: 12, 13, 14. The probe oligonucleotide may be labeled with a detectable marker, for example a fluorescence resonance energy transfer pair.

In another embodiment, a method of detecting a target RNA sequence of E. coli O157:H7 strains in a sample is provided. The method includes (a) reverse transcribing the E. coli O157:H7 strains target RNA in the presence of a reverse transcriptase activity and the reverse amplification primer to produce a target cDNA of the target RNA; (b) amplifying the target cDNA sequence to produce an increased number of copies of the target nucleic acid, the amplification including hybridizing a first primer including the sequence of SEQ ID NO: 16, 3, 4, or 6 and a second primer including the sequence of SEQ ID NO: 7, 8, 9, 10, or 11 to the target cDNA to obtain a hybridized product of the target nucleic acid and the primers, and extending the first and the second primers of the hybridized product using a template-dependent nucleic acid polymerase to produce an extended primer product; (c) hybridizing the target nucleic acid to at least one probe oligonucleotide which is substantially complimentary to the target cDNA to obtain a hybridized product of the target nucleic acid:probe oligonucleotide, wherein the probe comprises a DNA sequence and an RNA sequence and is coupled to a detectable marker; (d) contacting the hybridized product of the target nucleic acid:probe oligonucleotide with an RNase H to cleave the probe; and (e) detecting an increase in the emission of a signal from the detectable marker on the probe, wherein the increase in signal indicates the presence of the E. coli O157:H7 target RNA in the sample. The first primer may have the sequence of SEQ ID NO: 1, 2, 3, 4, 5, or 6. The probe oligonucleotide may have the oligonucleotide of SEQ ID NOs: 17 or 18. The probe oligonucleotide may be one of oligonucleotides of SEQ ID NOs: 12, 13, 14. The probe oligonucleotide may be labeled with a detectable marker, for example a fluorescence resonance energy transfer pair.

### BRIEF DESCRIPTION OF THE DRAWINGS

FIG. 1(A) shows amplification curves obtained by real-time polymerase chain reaction (PCR) of E. coli O157:H7 strains using a kit according to an embodiment of the present invention, and FIG. 1(B) shows Cp values determined from the data in FIG. 1(A);

FIG. 2 shows amplification curves obtained by real-time PCR of 63 different types of E. coli O157:H7 strains using a kit according to an embodiment of the present invention;

FIGS. 3(A)-3(C) show the amplication curves obtained by real-time of 59 non-E. coli O157:H7 strains, compared with O157:H7 strain, showing the kit and method according to an embodiment provides highly accurate results. The fifty-nine strains were tested in three divided tests; and

FIGS. 4(A) and 4(B) show the amplication curves obtained by real-time of E. coli O157:H7 strain using various combinations of primers and probes, in which FIG. 4(A) shows fluorescence curves and 4(B) shows the Cp values.

### DETAILED DESCRIPTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

The term “amplification” used herein refers to any process for increasing the number of copies of nucleotide sequences. Nucleic acid amplification describes a process whereby nucleotides are incorporated into nucleic acids, for example, DNA or RNA.

The term “nucleotide” used herein refers to a base-sugar-phosphate combination. Nucleotides are the monomeric units of nucleic acids, for example, DNA or RNA. The term “nucleotide” includes ribonucleoside triphosphates, such as rATP, rCTP, rGTP, or rUTP, and deoxy-ribonucleotide triphosphates, such as dATP, dCTP, dGTP, or dTTP.

The term “nucleoside” used herein refers to a base-sugar combination, i.e., a nucleotide lacking phosphate moieties. The terms “nucleoside” and “nucleotide” are used interchangeably in the field. For example, the nucleotide deoxyuridine, dUTP, is a deoxynucleoside triphosphate. It serves as a DNA monomer, for example, being dUMP or deoxyuridine monophosphate, after being inserted into DNA. In this regard, even though no dUTP moiety is present in the result DNA, dUTP may be considered as having been inserted.

The term “polymerase chain reaction (PCR)” generally refers to an amplification method for increasing the number of copies of target nucleic acid(s) in a sample. The procedure is described in detail in U.S. Pat. Nos. 4,683,202, 4,683,195, 4,800,159, and 4,965,188, the contents of which are incorporated herein in their entirety. The sample may include a single nucleic acid or multiple nucleic acids. In general, PCR involves incorporating at least two extendible primer nucleic acids into a reaction mixture containing target nucleic acid(s). The primers are complementary to opposite strands of a double-stranded target sequence. The reaction mixture is subjected to thermal cycling in the presence of a nucleic acid polymerase and nucleic acid monomers, for example, in the presence of dNTP's and/or rNTP's, to amplify the target nucleic acid by extension of the primers. In general, the thermal cycling may involve: annealing to hybridize the primer and target nucleic acid; extending the primers using a nucleic acid polymerase; and denaturating the hybridized primer extension product and the target nucleic acid. The term “reverse transcriptase-PCR (RT-PCR)” is a PCR that uses an RNA template and a reverse transcriptase, or an enzyme having reverse transcriptase activity, to first generate a single stranded cDNA molecule prior to the multiple cycles of DNA-dependent DNA polymerase primer extension. The term “multiplex PCR” refers to PCRs that produce more than two amplified target products in a single reaction, typically by the inclusion of more than two primers.

The term “nucleic acid” used herein refers to a polymer including more than two nucleotides. The term “nucleic acid” is used interchangeably with “polynucleotide” or “oligonucleotide”. Nucleic acids include DNA and RNA. The structure of nucleic acids may be double-stranded and/or single-stranded.

The term “nucleic acid analog” used herein refers to a nucleic acid that contains at least one nucleotide analog and/or at least one phosphate ester analog and/or at least one pentose sugar analog. Examples of nucleic acid analogues include nucleic acids in which the phosphate ester and/or sugar phosphate ester linkages are replaced with other types of linkages, such as N-(2-aminoethyl)-glycine amides and other amides. Nucleic acid analogs refer to a nucleic acid that contains at least one nucleotide analog and/or at least one phosphate ester analog and/or at least one pentose sugar analog and may form a double helix by hybridization.

The terms “annealing” and “hybridization” used herein are interchangeable and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The “nucleotide” used herein is a double-stranded or a single-stranded deoxyribonucleotide or ribonucleotide and includes nucleotide analogues unless otherwise stated.

The “primer” used herein is a single-stranded oligonucleotide functioning as an origin of polymerization of template DNA under appropriate conditions (i.e., 4 types of different nucleoside triphosphates and polymerases) at a suitable temperature and in a suitable buffer solution. The length of the primer may vary according to various factors, for example, temperature and the use of the primer, but the primer generally has 15 to 30 nucleotides. Generally, a short primer may form a sufficiently stable hybrid complex with its template at a low temperature. The “forward primer” and “reverse primer” are primers respectively binding to a 3′ end and a 5′ end of a specific region of a template that is amplified by PCR. The sequence of the primer is not required to be completely complementary to a part of the sequence of the template. The primer may have sufficient complementarity to be hybridized with the template and perform intrinsic functions of the primer. Thus, a primer set according to an embodiment is not required to be completely complementary to the nucleotide sequence as a template. The primer set may have sufficient complementarity to be hybridized with the sequence and perform intrinsic functions of the primer. The primer may be designed based on the nucleotide sequence of a polynucleotide as a template, for example, using a program for designing primers (PRIMER 3 program). Meanwhile, a primer according to an embodiment may be hybridized or annealed to a part of a template to form a double-strand. Conditions for hybridizing nucleic acid suitable for forming the double-stranded structure are disclosed by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985). For example, the primer may include at least 10, or at least 15 consecutive nucleotides of any one of the nucleotide sequences of SEQ ID NOS: 1 to 12. The primer may also be a nucleotide having any one of the nucleotide sequences of SEQ ID NOS: 3, 6-12 or 16. In an embodiment, the primer may be one of the sequences of SEQ ID NOS: 1-12.

The term “probe” used herein refers to a nucleic acid having a sequence complementary to a target nucleic acid sequence and capable of hybridizing to the target nucleic acid to form a duplex. The sequence of the probe may be fully or completely complementary to the target nucleic acid sequence. The probe may be labeled so that the target nucleic acid may be detected simultaneously with PCR.

The terms “target nucleic acid” or “target sequence” used herein includes a full length or a fragment of a target nucleic acid that may be amplified and/or detected. A target nucleic acid may be present between two primers that are used for amplification.

The term “hybrid oligonucleotide” used herein with regard to an oligonucleotide means an oligonucleotide molecule which contains a DNA and an RNA portion within a single molecule. The hybrid oligonucleotide may contain more than one DNA portion and one RNA portion, for example a DNA-RNA, RNA-DNA, or DNA-RNA-DNA oligonucleotide.

In embodiments, an oligonucleotide set for detecting E. coli O157:H7 includes (i) a first primer having the oligonucleotide of SEQ ID NOS: 16, 3, 4, or 6 and (ii) a second primer having the oligonucleotide of SEQ ID NOS: 7, 8, 9, 10, or 11. The oligonucleotide set may further include a probe selected from SEQ ID NOS: 17 or 18. In an embodiment, the first primer may be one of SEQ ID NO: 1, 2, 3, 4, 5, or 6. In an embodiment, the probe may be one of SEQ ID NO: 12, 13, or 14. In an embodiment, these oligonucleotides may have at least 10, or at least 15 consecutive sequences of SEQ ID NO: 1-12.

The primer pair of a first primer and a second primer according to an embodiment are specific to a part of I fragment of E. coli O157:H7. The I fragment is located at 312001-315400 of E. coli O157:H7 genome (GenBank: AE005174.2.). Sequence of the I fragment is shown as SEQ ID NO: 15.

In one embodiment, the probe may have a DNA-RNA-DNA hybrid structure. The probe may be a nucleic acid or a nucleic acid analog. The probe also may be a protected nucleic acid. For example, a DNA or RNA portion of the probe may be partially methylated to be resistant to degradation by an RNA-specific enzyme, for example, an RNase H.

The probe may be modified. For example, the base portion of the probe may be partially or fully methylated. Such modifications may inhibit enzymatic or chemical degradation. The 5′ end or 3′ end —OH group of the nucleic acid probe may be blocked. The 3′ end OH group of the nucleic acid probe may be blocked, thus being rendered incapable of extension by a template-dependant nucleic acid polymerase.

The probe may have a detectable label. The detectable label may be any chemical moiety detectable by any method known in the field. Examples of detectable labels include any moiety detectable by spectroscopy, photochemistry, or by biochemical, immunochemical or chemical means. A suitable method of labeling the nucleic acid probe may be selected according to the type of the label and the positions of the label and probe. Examples of labels include enzymes, enzyme substrates, radioactive substance, fluorescent dyes, chromophores, chemiluminescent labels, electrochemical luminescent label, ligands having specific binding partners, and other labels that interact with each other to increase, vary or reduce the intensity of a detection signal. These labels are durable throughout the thermal cycling for PCR.

The detectable label may be a fluorescence resonance energy transfer (FRET) pair. The detectable label is a FRET pair including a fluorescent donor and a fluorescent acceptor separated by an appropriate distance, and in which donor fluorescence emission is quenched by the acceptor. However, when the donor-acceptor pair is dissociated by cleavage, donor fluorescence emission is enhanced. A donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Examples of donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. In addition, an example of the detectable label is a non-fluorescent acceptor that will quench a wide range of donors. Other examples of appropriate donor-acceptor FRET pairs will be known to those of skill in the art.

In an embodiment, the probe may be present as a soluble form or free form in a solution. In another embodiment, the probe can be immobilized to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include polystyrene, avidin coated polystyrene beads cellulose, nylon, acrylamide gel and activated dextran, controlled pore glass (CPG), glass plates and highly cross-linked polystyrene. These solid supports are preferred for hybridization and diagnostic studies because of their chemical stability, ease of functionalization and well defined surface area. Solid supports such as controlled pore glass (500 \acute{Å}, 1000 \acute{Å}) and non-swelling high cross-linked polystyrene (1000 \acute{Å}) are particularly preferred in view of their compatibility with oligonucleotide synthesis.

The probe may be attached to the solid support in a variety of manners. For example, the probe may be attached to the solid support by attachment of the 3′ or 5′ terminal nucleotide of the probe to the solid support. However, the probe may be attached to the solid support by a linker which serves to separate the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and is completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the hybridization probe is immobilized on a solid support. The oligonucleotide probe is contacted with a sample of nucleic acids under conditions favorable for hybridization. In an unhybridized state, the fluorescent label is quenched by the acceptor. Upon hybridization to the target, the fluorescent label is separated from the quencher and the fluorescence emission is enhanced.

Immobilization of the hybridization probe to the solid support also enables the target sequence hybridized to the probe to be readily isolated from the sample. In later steps, the isolated target sequence may be separated from the solid support and processed (e.g., purified, amplified) according to methods well known in the art depending on the particular needs of the researcher.

The oligonucleotides according to an embodiment may be used for amplification and detection of target nucleic acids. The amplification may include extending the primers using a template-dependent polymerase, which results in the formation of PCR fragment or amplicon. The amplification can be accomplished by any method selected from the group consisting of Polymerase Chain Reaction or by using amplification reactions such as Ligase Chain Reaction, Self-Sustained Sequence Replication, Strand Displacement Amplification, Transcriptional Amplification System, Q-Beta Replicase, Nucleic Acid Sequence Based Amplification (NASBA), Cleavage Fragment Length Polymorphism, Isothermal and Chimeric Primer-initiated Amplification of Nucleic Acid, Ramification-extension Amplification Method or other suitable methods for amplification of nucleic acid. The amplification may include simultaneous real-time detection of target nucleic acids

The term “PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or a mixture thereof in any concentration ratio. A PCR fragment can be 100-500 nucleotides or more in length.

An amplification “buffer” is a compound added to an amplification reaction which modifies the stability and/or activity of one or more components of the amplification reaction by regulating the amplification reaction. The buffering agents of the invention are compatible with PCR amplification and RNase H cleavage activity. Examples of buffers include, but are not limited to, HEPES ((4-(2-hydroxyethyl)-1-piperazineethanesulfonic acid), MOPS (3-(N-morpholino)-propanesulfonic acid), and acetate or phosphate containing buffers and the like. In addition, PCR buffers may generally contain up to about 70 mM KCl and about 1.5 mM or higher MgCl₂, and about 50-200 μM each of dATP, dCTP, dGTP and dTTP. The buffers of the invention may contain additives to optimize efficient reverse transcriptase-PCR or PCR reactions.

An additive is a compound added to a composition which modifies the stability and/or activity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. Additives may be optionally added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, a “thermostable polymerase” is an enzyme that is relatively stable to heat and eliminates the need to add enzyme prior to each PCR cycle. Non-limiting examples of thermostable polymerases may include polymerases isolated from the thermophilic bacteria Thermus aquaticus (Taq polymerase), Thermus thermophilus (Tth polymerase), Thermococcus litoralis (Tli or VENT polymerase), Pyrococcus furiosus (Pfu or DEEPVENT polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME polymerase) Thermotoga neapolitana (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include AmpliTaq, AmpliTaq Stoffel fragment, SuperTaq, SuperTaq plus, LA Taq, LApro Taq, and EX Taq.

One of the most widely used techniques to study gene expression exploits first-strand cDNA for mRNA sequence(s) as template for amplification by the PCR. This method, often referred to as reverse transcriptase—PCR, exploits the high sensitivity and specificity of the PCR process and is widely used for detection and quantification of RNA.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” reverse transcriptase PCR methods use a common buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV reverse transcriptase and Taq DNA Polymerase wherein the initial 65° C. RNA denaturation step was omitted.

The first step in real-time, reverse-transcription PCR is to generate the complementary DNA strand using one of the template specific DNA primers. In traditional PCR reactions this product is denatured, the second template specific primer binds to the cDNA, and is extended to form duplex DNA. This product is amplified in subsequent rounds of temperature cycling. To maintain the highest sensitivity it is important that the RNA not be degraded prior to synthesis of cDNA. The presence of RNase H in the reaction buffer will cause unwanted degradation of the RNA:DNA hybrid formed in the first step of the process because it can serve as a substrate for the enzyme. There are two major methods to combat this issue. One is to physically separate the RNase H from the rest of the reverse-transcription reaction using a barrier such as wax that will melt during the initial high temperature DNA denaturation step. A second method is to modify the RNase H such that it is inactive at the reverse-transcription temperature, typically 45-55° C. Several methods are known in the art, including reaction of RNase H with an antibody, or reversible chemical modification. For example, a hot start RNase H activity as used herein can be an RNase H with a reversible chemical modification produced after reaction of the RNase H with cis-aconitic anhydride under alkaline conditions. When the modified enzyme is used in a reaction with a Tris based buffer and the temperature is raised to 95° C. the pH of the solution drops and RNase H activity is restored. This method allows for the inclusion of RNase H in the reaction mixture prior to the initiation of reverse transcription.

Additional examples of RNase H enzymes and hot start RNase H enzymes that can be employed in the invention are described in U.S. Patent Application No. 2009/0325169 to Walder et al., the content of which is incorporated herein in its entirety.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by real-time detection in combination with these reverse transcriptase-PCR techniques has enabled accurate and precise determination of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“Taq-Man”) or endonuclease assay (sometimes referred to as, “CataCleave”), discussed below.

Post-amplification amplicon detection is both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan probes (e.g., U.S. Pat. Nos. 5,210,015 and 5,487,972), and CataCleave probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded, the distance between the donor and acceptor chromophores increases such that FRET is reversed and donor emission can be monitored using specialized instrumentation. TaqMan and CataCleave technologies differ from the molecular beacon in that the FRET probes employed are cleaved such that the donor and acceptor chromophores become sufficiently separated to reverse FRET.

TaqMan technology employs a single stranded oligonucleotide probe that is labeled at the 5′ end with a donor chromophore and at the 3′ end with an acceptor chromophore. The DNA polymerase used for amplification must contain a 5′→3′ exonuclease activity. The TaqMan probe binds to one strand of the amplicon at the same time that the primer binds. As the DNA polymerase extends the primer the polymerase will eventually encounter the bound TaqMan probe. At this time the exonuclease activity of the polymerase will sequentially degrade the TaqMan probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan works a specific amplicon can be detected only once for every cycle of PCR. Extension of the primer through the TaqMan target site generates a double stranded product that prevents further binding of TaqMan probes until the amplicon is denatured in the next PCR cycle.

U.S. Pat. No. 5,763,181, the content of which is incorporated herein by reference, describes another real-time detection method (referred to as “CataCleave”). CataCleave technology differs from TaqMan in that cleavage of the probe is accomplished by a second enzyme that does not have polymerase activity. The CataCleave probe has a sequence within the molecule which is a target of an endonuclease, such as a restriction enzyme or RNase. In one example, the CataCleave probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan probe and donor emission can be monitored. Cleavage and dissociation regenerates a site for further CataCleave binding. In this way it is possible for a single amplicon to serve as a target or multiple rounds of probe cleavage until the primer is extended through the CataCleave probe binding site.

In embodiments, the probe used in the method is a CataCleave probe. Examples of suitable CataCleave probes include oligonucleotides comprising the sequence of one of SEQ ID NOS: 17 or 18. In an embodiment, the probe is one of the sequences of SEQ ID NO: 12, 13, or 14.

The Sequences of SEQ ID NO: 1-14 and 16-18 are shown below in Table 1.

TABLE 1

| SEQ ID | | Identification of |
|---|---|---|
| NO: | Sequences | Primer/probe |
| 1 | TTAACGAGCTGTATGTCGTGAGAAT | O157-I-F |
| 2 | AACGAGCTGTATGTCGTGAGAATC | O157-I-F1 |
| 3 | CCCTCCAAATGAAATTCCAACA | O157-I-F2 |
| 4 | GGCTTTGTTGCAAGGCTATG | O157-I2-F |
| 5 | CGAGCTGTATGTCGTGAGAATC | O157-I3-F |
| 6 | CAAGCCTATTCAGAGCATGG | O157-I4-F |
| 7 | ATGGATCATCAAGCTCTAAGAAAGAAC | O157-I-R |
| 8 | AGTGTCGTCTGTATGGATCATCAAG | O157-I-R1 |
| 9 | CCTCAAGCGAAGATGCAAAAT | O157-I-R2 |
| 10 | TGGATCATCAAGCTCTAAGAAAGAAC | O157-I-R3 |
| 11 | GATTGCAACTGCTCATCAGG | O157-I2-R |
| 12 | ATAGGCTTrGrArArGCAGTGCA,wherein rG | O157-I-P1 |
| | and at positions 9-12 are reach | |
| | ibonucleotides. | |
| 13 | ATAGGCTTrGrArArGCAGTGCAT,wherein rG | O157-I-P2 |
| | and at positions 9-12 are reach | |
| | ibonucleotides. | |
| 14 | TCAGAGCATGrGrArArATAAAACTT, wherein | O157-I-P3 |
| | rG and at positions 11-14 are reach | |
| | ibonucleotides. | |
| 16 | X₁X₁X₂X₂CGAGCTGTATGTCGTGAGAATX₃ in | |
| | which X₁ at positions 1 and 2 are absence or T, | |
| | X₂ at positions 3 and 4 are absence or A, and X₃ | |
| | at position 26 is absence or C | |
| 17 | ATAGGCTTGAAGCAGTGCAX₁, wherein X1 is | |
| | absence or T, and at least 3 consecutive | |
| | nucleotides at positions 9-14 are a ribonucleotide | |
| 18 | TCAGAGCATGGAAATAAAACTT, wherein at least | |
| | 3 consecutive nucleotides at positions 10-14 are a | |
| | ribonucleotide | |

The probes of SEQ ID NO: 12, 13, 14, 17 or 18 may be coupled to a detectable marker at each of their 5′- and 3′-ends. In an embodiment, 5′-end is coupled to FAM (6-carboxyfluorescein) and 3′-end is coupled to Black Hole Quencher (BHQ) for short wavelength emission.

In embodiments, a kit for detecting E. coli O157:H7 in a sample includes the oligonucleotides described above.

The kit may further include a reagent for nucleic acid amplification. The reagent may further include at least one selected from the group consisting of dNTP's, rNTP's, a nucleic acid polymerase, a uracil N-glycosylase (UNG) enzyme, a buffer, and a cofactor (for example, Mg²⁺). The nucleic acid polymerase may be selected from the group consisting of a DNA polymerase, a RNA polymerase, and a reverse transcriptase. The nucleic acid polymerase may be thermostable. The nucleic acid polymerase may retain its activity at elevated temperatures, for example, at 95° C. or higher. Thermostable DNA polymerases may be isolated from heat-resistant bacteria selected from the group consisting of Thermus aquaticus, Thermus flavus, Thermus ruber, Thermus thermophilus, Bacillus stearothermophilus, Thermus lacteus, Thermus rubens, Thermotoga maritima, Thermococcus littoralis, and Methanothermus fervidus. An example of a thermostable DNA polymerase is a Taq polymerase. The Taq polymerase is known to have optimal activity at about 70° C.

When the probe is hybridized to a target DNA, the E. coli O157:H7 detection kit may further include a factor specifically cleaving the RNA portion of the DNA-RNA hybrid. The cleaving factor may be RNase H. The cleaving factor may cleave specifically or nonspecifically the RNA portion. A specific RNA cleaving factor may be RNase HI. A nonspecific RNA cleaving factor may be RNase HII. RNase H may hydrolyze RNA in the RNA-DNA hybrid. For RNase H activity, a divalent ion (for example, Mg²⁺, Mn²⁺) is required. The RNase H cleaves RNA 3′-O-P linkages to produce 3′-hydroxyl and 5′-phosphate end products. The RNase H may be selected from the group consisting of a Pyrococcus furiosus RNase HII, a Pyrococcus horikoshi RNase HII, a Thermococcus litoralis RNase HI, and a Thermus thermophilus RNase HI. The RNase H may be thermostable. For example, the RNase H may retain its activity during a denaturation process in PCR. The cleaving factor may be a reversibly modified form of a thermostable RNase HII, which is inactive in its modified form and active in its unmodified form, wherein the modification is a coupling of the RNase HII to a ligand, crosslinking of the RNase HII, or chemical reaction of an amino acid residue in the RNase HII, and wherein the enzymatic activity of the modified RNase HII is restored by heating or adjusting pH of a sample containing the RNase HII.

When the RNA portion of the probe that contains a DNA sequence and an RNA sequence is cleaved by the cleaving factor, dissociation may occur. Such dissociation may naturally occur due to a decrease in the melting temperature of the cleaved complex or may be facilitated by a factor, such as temperature elevation. Dissociated fragments may be detected by any method known in the field.

In embodiments, a method of detecting E. coli O157:H7 in a sample includes: (a) amplifying a target nucleic acid of E. coli O157:H7 in the sample to produce an increased number of copies of the target nucleic acid, the amplifying including hybridizing a first primer of SEQ ID NO: 16, 3, 4, or 6 and a second primer of SEQ ID NO: 7, 8, 9, 10, or 11 to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the target nucleic acid to at least one probe oligonucleotide which is capable of being hybridized to the target nucleic acid to obtain a hybridized product of the target nucleic acid:probe oligonucleotide, wherein the probe contains an RNA sequence and a DNA sequence, and is coupled to a detectable marker; (c) contacting the hybridized product of the target nucleic acid:probe with RNase H to cleave the probes, resulting in probe fragment dissociation from the target nucleic acid; and (d) detecting the detectable marker. In an embodiment, the first primer may include the sequence of SEQ ID NO: 1, 2, 3, 4, 5, or 6. In an embodiment, the probe may include the sequence of SEQ ID NO: 12, 13, or 14.

Amplification of a target sequence in a sample may be performed by using any nucleic amplification method, such as the Polymerase Chain Reaction or by using amplification reactions such as Ligase Chain Reaction, Self-Sustained Sequence Replication, Strand Displacement Amplification, Transcriptional Amplification System, Q-Beta Replicase, Nucleic Acid Sequence Based Amplification (NASBA), Cleavage Fragment Length Polymorphism, Isothermal and Chimeric Primer-initiated Amplification of Nucleic Acid, Ramification-extension Amplification Method or other suitable methods for amplification of nucleic acid.

In an embodiment, the method includes amplifying a target nucleic acid fragment of E. coli O157:H7, the amplifying including hybridizing a first primer of SEQ ID NO. 16, 3, 4, or 6 and a second primer of SEQ ID NO. 7, 8, 9, 10, or 11 to the target nucleic acid in the sample to obtain a hybridized product; and extending the primers of the hybridized product using a template-dependent nucleic acid polymerase to produce an extended primer product; hybridizing the target nucleic acid fragment to a probe of SEQ ID NO: 17 or 18 to obtain a hybridized product; contacting the hybridized product from the target nucleic acid fragment and the probe to a RNase H to cleave the probes, resulting in a probe fragment dissociating from the hybridized product; and detecting the detectable marker. In an embodiment, the first primer may be one of SEQ ID NO: 1, 2, 3, 4, 5, or 6. In an embodiment, the probe may be one of SEQ ID NO: 12, 13, or 14.

Hereinafter, the method will now be described in greater detail. The method includes amplifying a target nucleic acid fragment of E. coli O157:H7, including hybridizing a first primer including the sequence of SEQ ID NO: 16, 3, 4, or 6 and a second primer including the sequence of SEQ ID NO: 7, 8, 9, 10 or 11 to the target nucleic acid in the sample to obtain a hybridized product; and extending the primers of the hybridized product depending on a template using a template-dependent nucleic acid polymerase to produced an extended primer product. In an embodiment, the first primer may have the sequence of SEQ ID NO: 1, 2, 3, 4, 5, or 6. In an embodiment, the probe may have the sequence of SEQ ID NO: 12, 13, or 14.

The hybridization may be conducted in a liquid medium. A suitable liquid medium may be selected according to the requirement(s). The liquid medium may be, for example, water, a buffer, or a PCR mixture. Nonlimiting examples of buffers include PBS, Tris, MOPS and Tricine. The hybridization may be conducted under the conditions to facilitate the binding of the primer and the target nucleic acid, for example, at low temperatures and low salt concentrations. Those conditions to facilitate hybridization are known in the field. The target nucleic acid may be a single-stranded or double-stranded nucleic acid. For example, a double-stranded target nucleic acid may be denaturated into separate single strands. The target nucleic acid may be DNA or RNA.

The extending of the primer depending on a template refers to polymerization, which is known in the field. The nucleic acid polymerase may be thermostable.

The method of detecting E. coli includes hybridizing the target nucleic acid fragment to at least one probe selected from the group consisting of oligonucleotides of SEQ ID NOS: 17 or 18 to obtain a hybridized product. The probes described above may be used. In an embodiment, the probe has the sequence of SEQ ID NO: 12, 13, or 14. The probe may be labeled with a detectable marker, for example, an optically detectable marker. Detectable markers are known in the art and may be suitably selected. For example, a FRET pair may be used for the purpose of detecting the target sequence in an embodiment of the invention.

The hybridization may be conducted in a liquid medium. A suitable liquid medium may be selected according to the requirement(s). The liquid medium may be, for example, water, a buffer, or a PCR mixture. Nonlimiting examples of buffers include PBS, Tris, MOPS (3-(N-morpholino)propanesulfonic acid) and Tricine. The hybridization may be conducted under the conditions to facilitate the binding of the single-stranded nucleic acid probe and the target nucleic acid, for example, at low temperatures and low salt concentrations. Those conditions to facilitate hybridization are known in the field. The target nucleic acid may be a single-stranded or double-stranded nucleic acid. For example, a double-stranded target nucleic acid may be denaturated into separate single strands, as described above. The target nucleic acid may be DNA or RNA.

The method of detecting E. coli O157:H7 includes contacting the hybridized product from the target nucleic acid fragment and the probe to a RNase H to cleave the probe, resulting in probe fragment dissociating from the hybridized product; and The hybridized product and the RNase H may contact each other in a liquid medium. A suitable liquid medium may be selected according to the requirement(s). The liquid medium may be, for example, water, a buffer, or a PCR mixture. Nonlimiting examples of buffers include PBS, Tris, MOPS (3-(N-morpholino)propanesulfonic acid) and Tricine. The contact may be conducted under substantially the same conditions as PCR conditions or in a PCR mixture.

The RNase H may be RNase HI or RNase HII. The RNase H may hydrolyze RNA in the RNA-DNA hybrid. For RNase H activity, a divalent ion (for example, Mg²⁺, Mn²⁺) is required. The RNase H cleaves RNA 3′-O-P linkages to produce 3′-hydroxyl and 5′-phosphate end products. The RNase H may be selected from the group consisting of a Pyrococcus furiosus RNase HII, a Pyrococcus horikoshi RNase HII, a Thermococcus litoralis RNase HI, and a Thermus thermophilus RNase HI. The RNase H may be thermostable. For example, the RNase H may retain its activity during a denaturation process in PCR. The RNase H may be a reversibly modified form of a thermostable RNase HII, which is inactive in its modified form and active in its unmodified form, wherein the modification is a coupling of the RNase HII to a ligand, crosslinking of the RNase HII, or chemical modification of the RNase HII, and wherein the enzymatic activity of the modified RNase HII is restored by heating or adjusting the pH of a sample containing the RNase HII.

Such dissociation may naturally occur due to the binding force of the strands that is weaken by the cleavage or may be facilitated by a factor, such as temperature elevation. For example, the PCR mixture may include an RNase H enzyme that will specifically cleave the RNA sequence portion of a RNA-DNA duplex. After cleavage, the two halves of the probe dissociate from a target amplicon at the reaction temperature and diffuse into the reaction buffer. As the donor and acceptors separate, FRET is reversed and donor emission can be monitored. Cleavage and dissociation regenerates a site for further probe binding. In this way it is possible for a single amplicon to serve as a target or multiple rounds of probe cleavage until the primer is extended through the probe binding site.

The method of detecting E. coli O157:H7 includes detecting the probe nucleic acid fragment.

An exemplary protocol for detecting a target E. coli O157:H7 sequence may include the steps of providing a food sample or surface wipe, mixing the sample or wipe with a growth medium and incubating to increase the number or population of E. coli O157:H7 (“enrichment”), disintegrating E. coli cells (“lysis”), and subjecting the obtained lysate to amplification and detection of target Salmonella sequence. Food samples may include, but are not limited to, fish such as smoked salmon, dairy products such as milk and cheese, and liquid eggs, poultry, fruit juices, meats such as ground pork, pork, ground beef, or beef, or deli meat, vegetables such as spinach, or environmental surfaces such as stainless steel, rubber, plastic, and ceramic.

The limit of detection (LOD) for food contaminants is described in terms of the number of colony forming units (CFU) that can be detected in either 25 grams of solid or 25 mL of liquid food or on a surface of defined area. By definition, a colony-forming unit is a measure of viable bacterial numbers. Unlike indirect microscopic counts where all cells, dead and living, are counted, CFU measures viable cells. One CFU (one bacterial cell) will grow to form a single colony on an agar plate under permissive conditions. The United States Food Testing Inspection Service defines the minimum LOD as 1 CFU/25 grams of solid food or 25 mL of liquid food or 1 CFU/surface area.

In practice, it is impossible to reproducibly inoculate a food sample or surface with a single CFU and insure that the bacterium survives the enrichment process. This problem is overcome by inoculating the sample at either one or several target levels and analyzing the results using a statistical estimate of the contamination called the Most Probable Number (MPN). As an example, an E. coli culture can be grown to a specific cell density by measuring the absorbance in a spectrophotometer. Ten-fold serial dilutions of the target are plated on agar media and the numbers of viable bacteria are counted. This data is used to construct a standard curve that relates CFU/volume plated to cell density. For the MPN to be meaningful, test samples at several inoculum levels are analyzed. After enrichment and extraction a small volume of sample is removed for real-time analysis. The ultimate goal is to achieve a fractional recovery of between 25% and 75% (i.e. between 25% and 75% of the samples test positive in the assay using RT-PCR employing a CataCleave probe, which will be explained below). The reason for choosing these fractional recovery percentages is that they convert to MPN values of between 0.3 CFU and 1.375 CFU for 25 gram samples of solid food, 25 mL samples of liquid food, or a defined area for surfaces. These MPN values bracket the required LOD of 1 CFU/sample. With practice, it is possible to estimate the volume of diluted innoculum (based on the standard curve) to achieve these fractional recoveries.

According to an embodiment, the kit may further include a mixture including dATP, dCTP, dGTP, and dTTP; a DNA polymerase; RNase HII; and a buffer solution.

The DNA polymerase may be a thermally stable DNA polymerase obtained from Thermus aquaticus (Taq), Thermus thermophilus (Tth), Thermus filiformis, Thermus flavus, Thermococcus literalis, or Pyrococcus furiosus (Pfu). In addition, RNase H includes a thermally stable RNase H enzyme such as Pyrococcus furiosus RNase H II, Pyrococcus horikoshi RNase H II, Thermococcus litoralis RNase HI, or Thermus thermophilus RNase HI, but is not limited thereto. The buffer solution is added to amplification to change stability, activity and/or lifetime of at least one component involved in the amplification reaction by controlling the pH of the amplification reaction. The buffer solution is well known in the art and may be Tris, Tricine, MOPS, or HEPES, but is not limited thereto. The kit may further include a dNTP mixture (dATP, dCTP, dGTP, and dTTP) and a DNA polymerase cofactor. The primer set and probe may be packed in a single reaction container, strip, or microplate by using various methods known in the art.

According to another embodiment, there is provided a method of detecting E. coli O157:H7 strains, the method including: obtaining E. coli O157:H7 lysates from a sample; performing a real-time PCR by mixing the lysates and the kit; and; and identifying the existence of E. coli O157:H7 strains based on results of the real-time PCR.

The method of detecting E. coli O157:H7 strains will now be described in more detail.

The method includes obtaining E. coli O157:H7 lysates from a sample.

The method may be applied to a sample that is assumed to be infected with E. coli O157:H7 strains. The sample may include cultured cells and body fluids such as blood and saliva, and foods such as meat, dairy products, and drinks, but is not limited thereto. Since the lysates include DNA of E. coli O157:H7, the DNA may be used as a template of a subsequent real-time PCR. For example, the lysates may be obtained by adding E. coli O157:H7 in a solution including 1 mg/mL proteinase K, 0.3125 mg/mL sodium azide, 0.125% Triton X-100, and 12.5 mM Tris-HCl, pH 8.0. In addition, DNA may be extracted from the lysates using various methods known in the art and used as a template of a real-time PCR. The method of extracting DNA from the lysates is disclosed in detail by Joseph Sambrook, et al., Molecular Cloning, A Laboratory Manual, Cold Spring Harbor Laboratory Press, Cold Spring Harbor, N.Y. (2001), the contents of which are entirely incorporated herein by reference.

The method includes performing a real-time PCR by mixing the lysates and the kit.

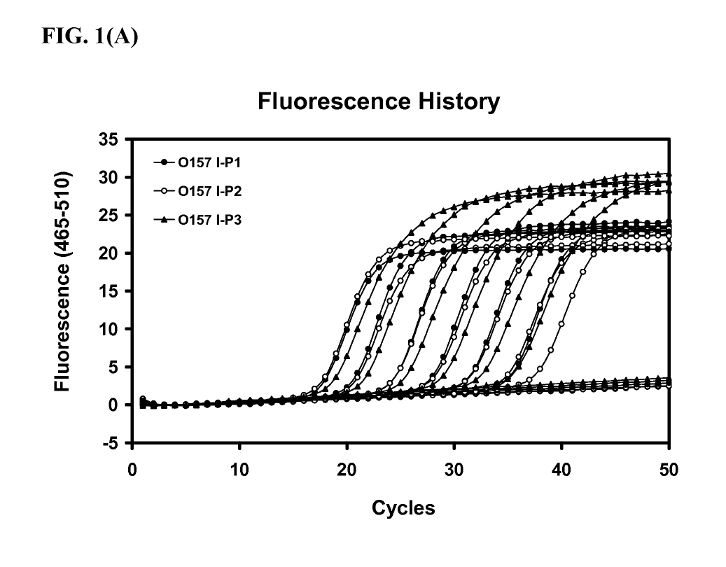

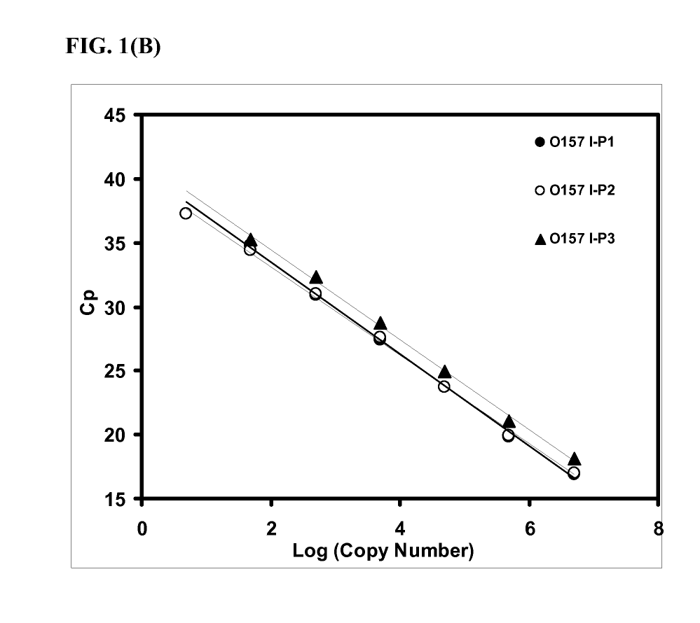

According to an embodiment, the kit for detecting E. coli O157:H7 strains may be used by using various methods and by using various devices for real-time PCR that are known in the art. The real-time PCR is a method of detecting fluorescence that is emitted in every cycle of PCR by a DNA polymerase and based on the FRET principle and quantifying the fluorescence in real-time using a device equipped with a thermal cycler and a spectrofluorophotometer. Using the real-time PCR, specific amplification products are distinguished from non-specific amplification products, and results of analysis may be automatically obtained without difficulty. The device used for the real-time PCR may include real-time PCR systems 7900, 7500, and 7300 (Applied Biosystems), Mx3000p (Stratagene), Chromo 4 (BioRad), and Roche Lightcycler 480, but is not limited thereto. While performing PCR, the real-time PCR device senses the fluorescence marker of the probe of amplified PCR products using laser beams to show peaks shown in FIG. 1.

In the method of detecting E. coli O157:H7 strains according to an embodiment, the real-time PCR may be performed using various methods that are known in the art. For example, an initial denaturation is performed at 95° C. for 10 minutes, and then a denaturation (at 95° C. for 15 seconds), and an annealing with primers and probes, and RNase HII reaction and elongation (at 60 or 63° C. for 20 seconds) are repeated 60 times. According to an embodiment, total 63 types of E. coli O157:H7 strains can be detected using the method.

The method includes identifying the existence of E. coli O157:H7 strains based on results of the real-time PCR.

The existence of E. coli O157:H7 strains may be identified by calculating a Cₚ value that is the number of cycles when the amount of the amplified PCR products reaches a predetermined level, based on the curve of the fluorescence marker labeled in the probe of the amplified PCR products obtained by the real-time PCR. If the Cₚ value is in the range of 10 to 50, or 15 to 45, it can be concluded that E. coli O157:H7 strains exist. Meanwhile, the Cₚ value may be automatically calculated by a program of the real-time PCR device.

Samples to be tested for the detection of E. coli O175:H7 are not limited, and may include meats (e.g., beef including ground beef), vegetables (e.g., spinach), fruit juices, and the like.

According to the kit for detecting E. coli O157:H7 strains and the method of detecting E. coli O157:H7 strains by using the kit, the results of the detection can be rapidly identified with a reduced number of copies of a sample in real-time.

The present invention will be described in further detail with reference to the following examples. These examples are for illustrative purposes only and are not intended to limit the scope of the invention.

### Example 1

### Preparation of Primer and Probe for Real-Time Detection of E. coli O157:H7

It was identified that primer used for real-time detection of E. coli O157:H7 has a nucleotide sequence capable of amplifying only a part of I fragment of E. coli O157:H7. The I fragment is located at 312001-315400 of E. coli O157:H7 genome (GenBank: AE005174.2.). The polynucleotide of the part of I fragment used in an embodiment is shown as SEQ ID NO: 15, which has 1720 nucleotides.

Table 1 below shows representative sequences of primers designed according to embodiments.

A CataCleave™ probe that specifically binds to a template of polymerase chain reaction (PCR) was prepared as the probe to detect the amount of PCR products that increases during real-time PCR in real-time. The 5′ end of the probe was labeled with 6-carboxyfluorescein (FAM) and the 3′ end of the probed was labeled with Black Hole Quencher (Integrated DNA Technologies, Coralville, Iowa). The determined primer and probe were synthesized by Roche Co., Ltd.

Table 1 discussed hereinbefore shows the representative sequences of probes designed. Probe sequences also show the markers coupled to the nucleotide.

### Example 2

### Amplification of E. coli O157:H7 Using Real-Time PCR

Total DNA of the E. coli O157:H7 which is used as a template for a real-time PCR was extracted using the following method. E. coli O157:H7 that was cultured and harvested (5 μL) was diluted in 45 μl of a lysing solution including 1 mg/mL proteinase K, 0.3125 mg/mL sodium azide, 0.125% Triton X-100, and 12.5 mM Tris-HCl, pH 8.0. The sample was cultured at 55° C. for 15 minutes, the proteinase K was inactivated at 95° C. for 10 minutes, and the sample was cooled to 4° C. The reactants were centrifuged to obtain a supernatant, and the supernatant or DNA extracted from the supernatant using various methods known in the art were added to a real-time PCR.

A mixture including 2 μL, of DNA and 23 μL, (out of 1656 μL) of a master mix was used for all real-time PCRs performed herein. The master mix includes 180 μL, of a 10×I buffer solution (10×I buffer is a HEPES-containing buffer (HEPES-KOH, MgCl₂, KCl, BSA, DMSO), 72 μL of 20 μM forward primer (SEQ ID NO: 1, 2, 3, or 4), 72 μL of 20 μM reverse primer (SEQ ID NO: 7, 8, 9, or 11), 14.4 μl of 25 μM CataCleave™ probe (SEQ ID NO: 12, 13, or 14), 72 μL of dNTP mix (2 mM dGTP, dCTP, dATP, and dTTP), 36 μL of Platinum® Taq DNA polymerase (Invitrogen), 14.4 μL of Pfu RNase HII, 7.2 μL of uracil DNA N-glycosylase, and 1188 μL of distilled water.

Uracil DNA N-glycosylase reaction was conducted at 37° C. for 10 minutes, and the resultant was denatured at 95° C. for 10 minutes. Then, real-time PCR was performed by repeating denaturation at 95° C. for 15 seconds and annealing with the primers and the CataCleave™ probe, reaction with RNase H, and elongation at 60° C. or 63° C. for 20 seconds 50 times. When the real-time PCR was completed, the resultant was cooled at 40° C. for 10 seconds. The reactions were performed using Roche Lightcycler 480, and PCR amplification was observed in real-time using the LightCycler 480 Software v1.5.0.

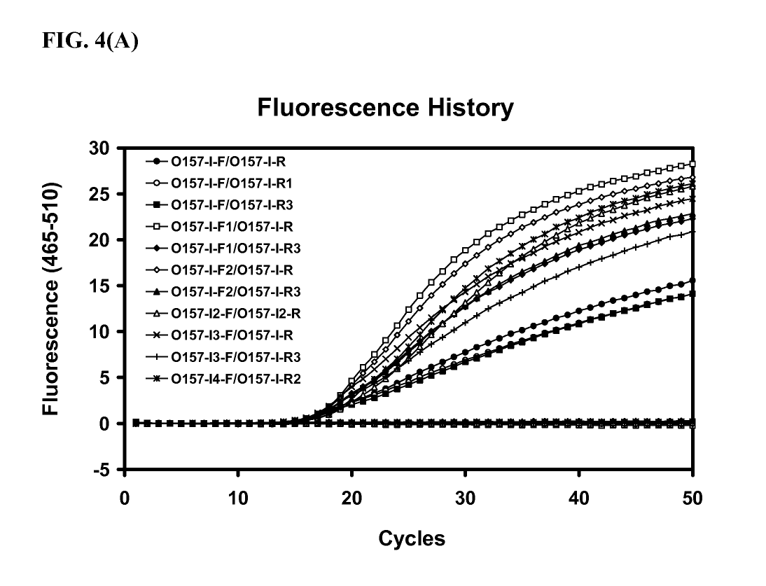

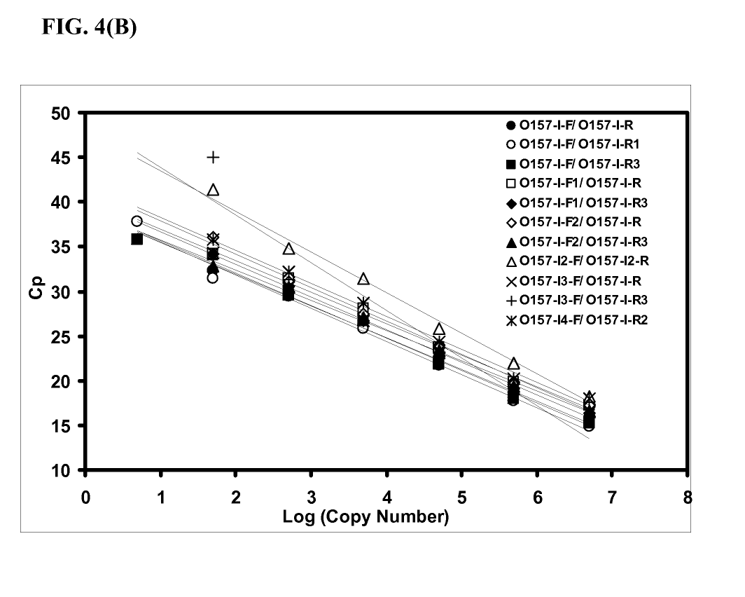

The results are shown in Table 2 and FIGS. 4A-4B. In Table 2 and FIGS. 4A-4B, the primers have the following the sequences:

O157-I-F: SEQ ID NO: 1

O157-I-F1: SEQ ID NO: 2

O157-I-F2: SEQ ID NO: 3

O157-I2-F: SEQ ID NO: 4

O157-I-R: SEQ ID NO: 7

O157-I-R1: SEQ ID NO: 8

O157-I-R3: SEQ ID NO: 10

O157-I2-R: SEQ ID NO: 11

TABLE 2

| | | | | | | O157- | O157- | O157- | O157- | O157- | O157- | O157- |
|---|---|---|---|---|---|---|---|---|---|---|---|---|
| | | O157-I-F/ | O157-I-F/ | O157-I-F/ | O157-I-F1/ | I-F1/ | I-F2/ | I-F2/ | I2-F/ | I3-F/ | I3-F/ | I4-F/ |
| Copy # | Log | O157-I-R | O157-I-R1 | O157-I-R3 | O157-I-R | O157-I-R3 | O157-I-R | O157-I-R3 | O157-I2-R | O157-I-R | O157-I-R3 | O157-I-R2 |
| 0.E+00 | | | | | | | | | | | | |
| 5.E+00 | 0.7 | | 37.75 | 35.83 | | | | | | | | |
| 5.E+01 | 1.7 | 32.26 | 31.41 | 34.09 | 34.14 | 34.23 | 36.00 | 32.85 | 41.37 | 34.33 | 45.00 | 35.76 |
| 5.E+02 | 2.7 | 30.40 | 29.38 | 29.59 | 31.40 | 30.62 | 31.12 | 30.44 | 34.82 | 30.56 | 31.35 | 32.16 |
| 5.E+03 | 3.7 | 26.14 | 25.87 | 26.70 | 28.06 | 26.66 | 27.49 | 27.05 | 31.39 | 26.66 | 26.67 | 28.63 |
| 5.E+04 | 4.7 | 22.26 | 21.67 | 21.86 | 23.72 | 23.08 | 23.96 | 23.36 | 25.87 | 23.05 | 23.15 | 24.30 |
| 5.E+05 | 5.7 | 18.16 | 17.68 | 18.01 | 19.80 | 19.06 | 19.82 | 19.33 | 22.01 | 19.11 | 18.72 | 20.20 |
| 5.E+06 | 6.7 | 15.61 | 14.91 | 15.23 | 17.23 | 16.36 | 17.25 | 16.58 | 18.24 | 16.42 | 15.93 | 17.95 |

| | | | | | | | | | | | |
|---|---|---|---|---|---|---|---|---|---|---|---|
| Slope | −3.54 | −3.70 | −3.63 | −3.53 | −3.65 | −3.75 | −3.38 | −4.56 | −3.64 | −5.34 | −3.69 |
| Y-Intercept | 39.00 | 39.22 | 39.34 | 40.56 | 40.31 | 41.68 | 39.14 | 48.10 | 40.32 | 49.21 | 42.01 |
| PCR | 91.7 | 86.2 | 88.5 | 91.9 | 88.0 | 84.8 | 97.6 | 65.7 | 88.1 | 54.0 | 86.5 |
| Efficiency | | | | | | | | | | | |

### Example 3

### Detection of E. coli O157:H7 Using Probes According to Embodiments

Real-time PCR of E. coli O157:H7 was performed using a primer set including O157-1-F1 (SEQ ID NO: 2) and O157-I-R (SEQ ID NO: 7) and three different probes of O157-I-P1 (SEQ ID NO: 12), O157-I-P2 (SEQ ID NO: 13), or O157-I-P3 (SEQ ID NO: 14)). The results are shown in FIGS. 1(A) and 1(B), which each show the amplification curves obtained by the real-time PCR, shown in fluorescence history and Cp values. In addition, Table 3 shows Cₚ values calculated based on the amplification curve of FIG. 1(A). In the experiment, the number of initial copies was 5,000,000. The results shown below indicate that amplification could be performed with 5 copies when the real-time PCR was performed using the primer set and the probe of O157-I-P2 (SEQ ID NO: 13). Meanwhile, fluorescence was not detected in a control to which distilled water was added instead of the DNA template.

TABLE 3

| Copy # | Log | O157 I-P1 | O157 I-P2 | O157 I-P3 |
|---|---|---|---|---|
| 0.E+00 | | | | |
| 5.E+00 | 0.7 | | 37.24 | |
| 5.E+01 | 1.7 | 34.76 | 34.43 | 35.28 |
| 5.E+02 | 2.7 | 30.86 | 30.95 | 32.30 |
| 5.E+03 | 3.7 | 27.44 | 27.60 | 28.68 |
| 5.E+04 | 4.7 | 23.74 | 23.75 | 24.94 |
| 5.E+05 | 5.7 | 19.85 | 19.92 | 21.06 |
| 5.E+06 | 6.7 | 16.94 | 16.95 | 18.17 |

| | | | |
|---|---|---|---|
| Slope | −3.60 | −3.47 | −3.51 |
| Y-Intercept | 40.69 | 40.09 | 41.50 |
| PCR Efficiency | 89.7 | 94.3 | 92.5 |

### Example 4

### Inclusivity Test of E. coli O157:H7

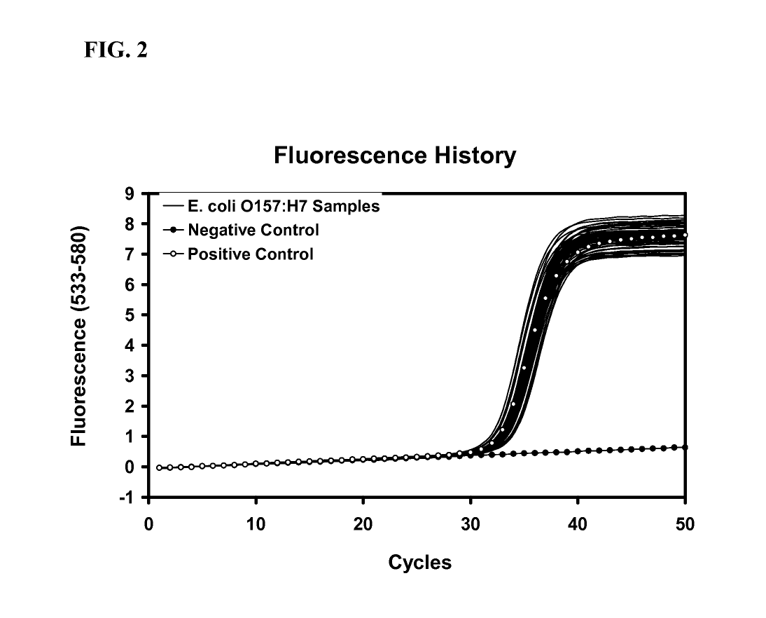

Inclusivity tests of 63 types of E. coli O157:H7 strains shown in Table 3 were conducted using the primer set and the probes used in Example 3. Real-time PCR was conducted using DNA having a concentration of 50,000 cfu/ml that is 10 times limit of detection (LOD) as a template. FIG. 2 shows an amplification curve obtained by the real-time PCR. In addition, Table 4 shows Cₚ values calculated based on the amplification curve of FIG. 2.

According to the results shown below, PCR products were detected in all of the 63 types of the E. coli O157:H7 strains (100% inclusivity) when the real-time PCR was performed using the primer set (SEQ ID NO: 2 and SEQ ID NO: 7) and the probe of O157-I-P2 (SEQ ID NO: 13), and an average Cₚ value was 32.57.

TABLE 4

| STA # | Serovar Name | Cp | RFU |
|---|---|---|---|
| 0070010002 | Escherichia coli O157:H7 | 33.47 | 7.095 |
| 0070010003 | Escherichia coli O157:H7 | 33.42 | 7.359 |
| 0070010004 | Escherichia coli O157:H7 | 32.72 | 7.722 |
| 0070010005 | Escherichia coli O157:H7 | 32.78 | 7.682 |
| 0070010006 | Escherichia coli O157:H7 | 32.64 | 7.389 |
| 0070010007 | Escherichia coli O157:H7 | 32.31 | 7.342 |
| 0070010008 | Escherichia coli O157:H7 | 31.74 | 7.543 |
| 0070010009 | Escherichia coli O157:H7 | 32.87 | 7.032 |
| 0070010010 | Escherichia coli O157:H7 | 32.06 | 7.373 |
| 0070010011 | Escherichia coli O157:H7 | 32.20 | 7.527 |
| 0070010012 | Escherichia coli O157:H7 | 32.27 | 7.800 |
| 0070010013 | Escherichia coli O157:H7 | 32.46 | 8.125 |
| 0070010014 | Escherichia coli O157:H7 | 31.87 | 7.713 |
| 0070010015 | Escherichia coli O157:H7 | 31.64 | 8.183 |
| 0070010016 | Escherichia coli O157:H7 | 32.16 | 7.659 |
| 0070010017 | Escherichia coli O157:H7 | 32.14 | 7.494 |
| 0070010018 | Escherichia coli O157:H7 | 33.23 | 7.254 |
| 0070010019 | Escherichia coli O157:H7 | 32.26 | 7.727 |
| 0070010020 | Escherichia coli O157:H7 | 33.07 | 7.766 |
| 0070010021 | Escherichia coli O157:H7 | 32.15 | 7.747 |
| 0070010022 | Escherichia coli O157:H7 | 32.43 | 7.924 |
| 0070010023 | Escherichia coli O157:H7 | 32.97 | 7.623 |
| 0070010024 | Escherichia coli O157:H7 | 32.98 | 8.065 |
| 0070010025 | Escherichia coli O157:H7 | 32.50 | 7.776 |
| 0070010026 | Escherichia coli O157:H7 | 32.52 | 7.003 |
| 0070010027 | Escherichia coli O157:H7 | 32.16 | 7.488 |
| 0070010028 | Escherichia coli O157:H7 | 32.26 | 7.518 |
| 0070010029 | Escherichia coli O157:H7 | 32.71 | 7.882 |
| 0070010030 | Escherichia coli O157:H7 | 31.79 | 7.802 |
| 0070010031 | Escherichia coli O157:H7 | 32.57 | 7.793 |
| 0070010032 | Escherichia coli O157:H7 | 32.54 | 7.716 |
| 0070010033 | Escherichia coli O157:H7 | 32.57 | 7.740 |
| 0070010034 | Escherichia coli O157:H7 | 32.64 | 6.956 |
| 0070010035 | Escherichia coli O157:H7 | 33.56 | 7.468 |
| 0070010036 | Escherichia coli O157:H7 | 33.66 | 8.055 |
| 0070010037 | Escherichia coli O157:H7 | 32.68 | 7.995 |
| 0070010038 | Escherichia coli O157:H7 | 32.77 | 7.776 |
| 0070010039 | Escherichia coli O157:H7 | 32.77 | 8.037 |
| 0070010040 | Escherichia coli O157:H7 | 32.62 | 7.587 |
| 0070010041 | Escherichia coli O157:H7 | 32.19 | 7.394 |
| 0070010042 | Escherichia coli O157:H7 | 32.63 | 7.140 |
| 0070010043 | Escherichia coli O157:H7 | 32.87 | 7.261 |
| 0070010044 | Escherichia coli O157:H7 | 32.54 | 7.772 |
| 0070010045 | Escherichia coli O157:H7 | 32.45 | 8.123 |
| 0070010046 | Escherichia coli O157:H7 | 32.46 | 8.123 |
| 0070010047 | Escherichia coli O157:H7 | 32.90 | 7.642 |
| 0070010048 | Escherichia coli O157:H7 | 32.47 | 7.667 |
| 0070010049 | Escherichia coli O157:H7 | 32.19 | 7.597 |
| 0070010050 | Escherichia coli O157:H7 | 32.92 | 6.996 |
| 0070010051 | Escherichia coli O157:H7 | 32.67 | 7.148 |
| 0070010052 | Escherichia coli O157:H7 | 33.00 | 7.598 |
| 0070010053 | Escherichia coli O157:H7 | 31.62 | 7.996 |
| 0070010054 | Escherichia coli O157:H7 | 32.85 | 7.610 |
| 0070010055 | Escherichia coli O157:H7 | 32.76 | 7.759 |
| 0070010056 | Escherichia coli O157:H7 | 32.33 | 7.615 |
| 0070010057 | Escherichia coli O157:H7 | 32.91 | 7.675 |
| 0070010058 | Escherichia coli O157:H7 | 32.53 | 7.092 |
| 0070010059 | Escherichia coli O157:H7 | 32.78 | 7.333 |
| 0070010060 | Escherichia coli O157:H7 | 32.62 | 8.023 |
| 0070010061 | Escherichia coli O157:H7 | 32.02 | 8.274 |
| 0070010062 | Escherichia coli O157:H7 | 32.21 | 7.933 |
| 0070010063 | Escherichia coli O157:H7 | 32.86 | 7.769 |
| 0070010064 | Escherichia coli O157:H7 | 32.70 | 7.782 |

| | | |
|---|---|---|
| Negative Control | | 0.637 |
| Positive Control (100 copies O157 I | 32.24 | 7.630 |
| fragment) | | |

The results show that the primers and probes according to embodiments of the invention allow detecting all of the tested E. coli O157:H7 strains with a high sensitivity.

### Example 5

### Exclusivity Test of E. coli O157:H7

Exclusivity tests of 59 types of non-E. coli O157:H7 strains shown in Table 4(A)-(C) were conducted using the primer set and probes used in Example 3.

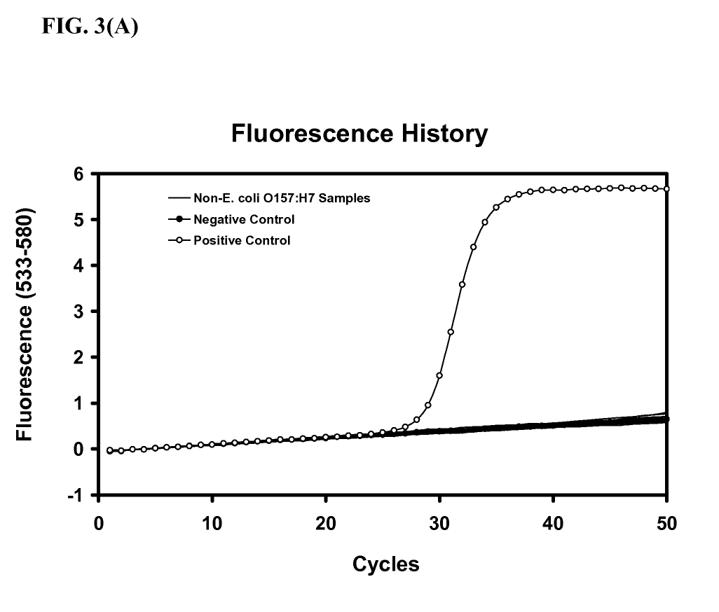

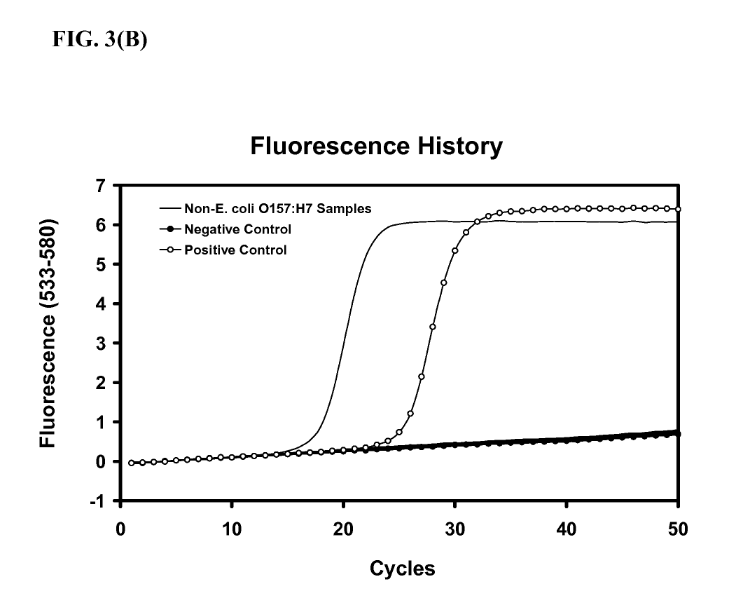

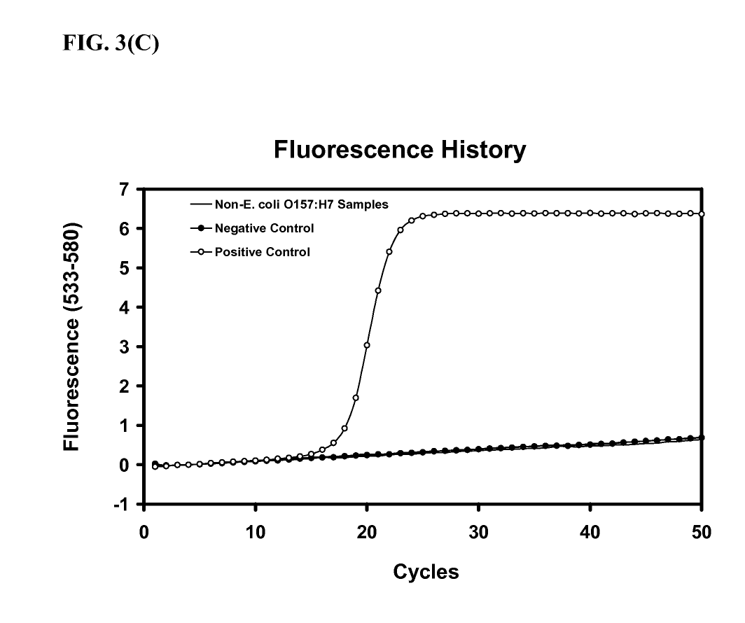

Real-time PCR was conducted using DNA from maximal density cultures (approximately 2×10⁹ cfu/mL) as a template. FIG. 3(A)-3(C) show amplification curves obtained by the real-time PCR for each of strains listed in Table 5(A)-(C), respectively. In addition, Tables 5(A)-(C) show Cₚ values calculated based on the amplification curve of FIG. 3(A)-3(C).

According to the results shown below, PCR products were not detected in the 58 types of the non-E. coli O157:H7 strains (98.3% exclusivity) when the real-time PCR was performed using the primer set (SEQ ID NO: 2 and SEQ ID NO: 7) and the probe of O157-I-P2 (SEQ ID NO: 13). PCR products were detected in the test of E. coli O55:H7 using the primer set and the probes. It is assumed that E. coli O55:H7 showed cross-reactivity since it is the ancestor of E. coli O157:H7 strains.

TABLE 5A

O157 I
(TYE563)

| STA # | Serovar Name | Cp | RFU |
|---|---|---|---|
| 0020010001 | Aeromonas caviae | | 0.644 |
| 0020020001 | Aeromonas hydrophila | | 0.652 |
| 0040010001 | Citrobacter amalonaticus | | 0.648 |
| 0040020001 | Citrobacter braakii | | 0.614 |
| 0040030001 | Citrobacter freundii | | 0.611 |
| 0040040001 | Citrobacter youngae | | 0.614 |
| 0050010001 | Edwardsiella tarda | | 0.603 |
| 0060010001 | Enterobacter aerogenes | | 0.580 |
| 0060020001 | Enterobacter cancerogenous | | 0.617 |
| 0060030001 | Enterobacter cloacae | | 0.645 |
| 0060040001 | Enterobacter intermedia | | 0.661 |
| 0060050001 | Enterobacter sakazakii | | 0.656 |
| 0070010001 | Escherichia coli | | 0.644 |
| 0070020001 | Escherichia fergusonii | | 0.605 |
| 0070030001 | Escherichia vulneris | | 0.620 |
| 0080010001 | Klebsiella pneumoniae | | 0.619 |
| 0090010001 | Morganella morganii | | 0.670 |
| 0100010001 | Proteus hauseri | | 0.706 |
| 0100020001 | Proteus mirabilis | | 0.666 |
| 0100030001 | Proteus vulgaris | | 0.673 |
| 0110010001 | Pseudomonas aeruginosa | | 0.634 |
| 0110020001 | Pseudomonas putida | | 0.794 |
| 0120010001 | Serratia marcescens | | 0.631 |
| 0130010001 | Shigella dysenteriae | | 0.617 |
| 0130020001 | Shigella flexneri | | 0.627 |
| 0130030001 | Shigella sonnei | | 0.647 |
| 0140010001 | Vibrio cholera | | 0.707 |
| 0150010001 | Yersinia enterocolitica | | 0.652 |

| | | |
|---|---|---|
| Campylobacter jejuni genomic DNA (1 mg) | | 0.766 |
| Negative control | | 0.651 |
| Positive Control (1 × 10³ copies O157 I | 28.77 | 5.667 |
| fragment) | | |

TABLE 5B

O157 I
(TYE563)

| STA # | Serovar Name | Cp | RFU |
|---|---|---|---|
| 0070040001 | Escherichia coli O157:H1 | | 0.743 |
| 0070050001 | Escherichia coli O157:H2 | | 0.755 |
| 0070060001 | Escherichia coli O157:H4 | | 0.718 |
| 0070070001 | Escherichia coli O157:H5 | | 0.726 |
| 0070080001 | Escherichia coli O157:H11 | | 0.763 |
| 0070090001 | Escherichia coli O157:H12 | | 0.754 |
| 0070100001 | Escherichia coli O157:H15 | | 0.781 |
| 0070110001 | Escherichia coli O157:H16 | | 0.753 |
| 0070120001 | Escherichia coli O157:H18 | | 0.775 |
| 0070130001 | Escherichia coli O157:H19 | | 0.763 |
| 0070150001 | Escherichia coli O157:H29 | | 0.766 |
| 0070160001 | Escherichia coli O157:H42 | | 0.675 |
| 0070170001 | Escherichia coli O157:H43 | | 0.680 |
| 0070190001 | Escherichia coli O157:H45 | | 0.711 |
| 0070200001 | Escherichia coli O157:HNM | | 0.708 |
| 0070210001 | Escherichia coli O157:HN | | 0.707 |
| 0070230001 | Escherichia coli O55:H7 | 17.88 | 6.078 |
| 0070240001 | Escherichia coli O91:HNM | | 0.676 |
| 0070250001 | Escherichia coli O111:H12 | | 0.760 |
| 0070270001 | Escherichia coli O117:H4 | | 0.739 |
| 0070280001 | Escherichia coli O103:H2 | | 0.785 |
| 0070290001 | Escherichia coli O115:HNM | | 0.731 |
| 0070300001 | Escherichia coli O118:H12 | | 0.704 |
| 0070310001 | Escherichia coli O121:HNM | | 0.775 |
| 0070320001 | Escherichia coli O142:HNM | | 0.800 |
| 0070330001 | Escherichia coli O145:HNM | | 0.706 |
| 0070340001 | Escherichia coli O146:H21 | | 0.694 |
| 0070350001 | Escherichia coli O163:H19 | | 0.739 |

| | | |
|---|---|---|
| Negative Control | | 0.687 |
| Positive Control (10000 copies O157 I | 25.36 | 6.394 |
| fragment) | | |

TABLE 5C

O157 I
(TYE563)

| STA # | Serovar Name | Cp | RFU |
|---|---|---|---|
| 0070220001 | Escherichia coli O26:H11 | | 0.692 |
| 0070260001 | Escherichia coli O111:H8 | | 0.633 |

| | | |
|---|---|---|
| TSB only | | 0.684 |
| Positive Control (1e7 copies O157 I | 17.74 | 6.293 |
| fragment) | | |

According to the results of Examples 1 to 5, E. coli O157:H7 strains can be efficiently detected with a reduced amount of samples using the primer sets and probes according to an embodiment. Thus time and effort for detecting E. coli O157:H7 strains can be reduced.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

What is claimed is:

1. A composition comprising:
a first oligonucleotide comprising at least 10 consecutive nucleotides of the sequence of SEQ ID NO: 16, 3, 4, or 6, and
a second oligonucleotide comprising at least 10 consecutive nucleotides of the sequence of SEQ ID NO: 7, 8, 9, 10, or 11.

2. The composition according to claim 1, further comprising a third oligonucleotide comprising a DNA sequence and an RNA sequence, said third oligonucleotide being the sequence of SEQ ID NO: 17 or SEQ ID NO: 18:
ATAGGCTTGAAGCAGTGCAX₁ (SEQ ID NO: 17), wherein X₁ is absence or T, and at least 3 consecutive nucleotides at positions 9-14 are a ribonucleotide,
TCAGAGCATGGAAATAAAACTT (SEQ ID NO: 18), wherein at least 3 consecutive nucleotides at positions 10-14 are a ribonucleotide.

3. The composition according to claim 2, wherein the third oligonucleotide is one or more selected from the group consisting of the oligonucleotides of SEQ ID NOs: 12-14:
ATAGGCTTrGrArArGCAGTGCA (SEQ ID NO: 12), wherein rG and rA at positions 9-12 are each ribonucleotides,
ATAGGCTTrGrArArGCAGTGCAT (SEQ ID NO: 13), wherein rG and rA at positions 9-12 are each ribonucleotides, and
TCAGAGCATGrGrArArATAAAACTT (SEQ ID NO: 14), wherein rG and rA at positions 11-14 are each ribonucleotides.

4. The composition according to claim 2, wherein the third oligonucleotide is labeled with a detectable marker.

5. The composition according to claim 4, wherein the third oligonucleotide is labeled with a fluorescence resonance energy transfer (FRET) pair.

6. The composition according to claim 1, wherein the first oligonucleotide has the sequence selected from the group of the sequences of SEQ ID NOS: 1, 2, 3, 4, 5, and 6.

7. The composition according to claim 6, comprising the first oligonucleotide of SEQ ID NO. 2, the second oligonucleotide of SEQ ID NO. 7, and the third oligonucleotide of SEQ ID NO. 13.

8. A kit for detecting E. coli O157:H7 in a sample, the kit comprising
(a) a first primer comprising at least 10 consecutive nucleotides of the sequence of SEQ ID NOS: 16, 3, 4, or 6;
(b) a second primer comprising at least 10 consecutive nucleotides of the sequence of SEQ ID NOS: 7, 8, 9, 10, or 11; and
(c) a probe comprising an RNA sequence and a DNA sequence that are substantially complimentary to a target gene of E. coli. O157:H7, and coupled to a detectable label.

9. The kit according to claim 8, wherein the target E. coli. O157:H7 is a gene of SEQ ID NO: 15 or a fragment thereof.

10. The kit according to claim 8, further comprising
(d) an amplifying activity for a PCR amplification of the target DNA sequence to produce a E. coli O157:H7 PCR fragment; and
(e) an RNase H activity.

11. The kit according to claim 10, further comprising positive, internal, and negative controls.

12. The kit according to claim 11, further comprising uracil-N-glycosylase.

13. The kit according to claim 8, wherein the detectable marker is a fluorescent label.

14. The kit according to claim 13, wherein the probe is labeled with a FRET pair.

15. The kit according to claim 8, wherein the probe is immobilized to a solid support.

16. The kit according to claim 8, wherein the probe is in free form in a solution.

17. The kit according to claim 8, which further comprises an amplification buffer.

18. The kit according to claim 8, which further comprises an amplifying polymerase activity.

19. The kit according to claim 10, wherein the RNase H activity is the activity of a thermostable RNase H.

20. The kit according to claim 11, wherein the RNase H activity is a hot start RNase H activity.

21. The kit according to claim 8, wherein the probe comprises the sequence of SEQ ID NO: 17 or SEQ ID NO: 18:
ATAGGCTTGAAGCAGTGCAX₁ (SEQ ID NO: 17), wherein X₁ is absence or T, and at least 3 consecutive nucleotides at positions 9-14 are a ribonucleotide,
TCAGAGCATGGAAATAAAACTT (SEQ ID NO: 18), wherein at least 3 consecutive nucleotides at positions 10-14 are a ribonucleotide.

22. The kit according to claim 21, wherein the third oligonucleotide is one or more selected from the group consisting of the oligonucleotides of SEQ ID NOs: 12-14:
ATAGGCTTrGrArArGCAGTGCA (SEQ ID NO: 12), wherein rG and rA at positions 9-12 are each ribonucleotides,
ATAGGCTTrGrArArGCAGTGCAT (SEQ ID NO: 13), wherein rG and rA at positions 9-12 are each ribonucleotides, and
TCAGAGCATGrGrArArATAAAACTT (SEQ ID NO: 14), wherein rG and rA at positions 11-14 are each ribonucleotides.

23. A method of detecting E. coli O157:H7 in a sample, the method comprising:
(a) amplifying a target nucleic acid of E. coli O157:H7 in the sample to produce an increased number of copies of the target nucleic acid, the amplifying including hybridizing a first primer comprising at least 10 consecutive nucleotide of the sequence of SEQ ID NO: 16, 3, 4, or 6 and a second primer comprising at least 10 consecutive nucleotides of the sequence of SEQ ID NO: 7, 8, 9, 10, or 11 to the target nucleic acid in the sample to obtain a hybridized product of the target nucleic acid and the primers, and extending the first and the second primers of the hybridized product using a template-dependent nucleic acid polymerase to produce an extended primer product;
(b) hybridizing the target nucleic acid to at least one probe oligonucleotide which is capable of being hybridized to the target nucleic acid to obtain a hybridized product of the target nucleic acid:probe oligonucleotide, wherein the probe comprises a DNA sequence and an RNA sequence and is coupled to a detectable label;
(c) contacting the hybridized product of the target nucleic acid:the probe oligonucleotide to an RNase H to cleave the probes; and
(d) detecting an increase in the emission of a signal from the label on the probe, wherein the increase in signal indicates the presence of the E. coli O157:H7 nucleic acid in the sample.

24. The method according to claim 23, wherein the probe oligonucleotide is the oligonucleotide of SEQ ID NO: 17 or 18:
ATAGGCTTGAAGCAGTGCAX₁ (SEQ ID NO: 17), wherein X₁ is absence or T, and at least 3 consecutive nucleotides at positions 9-14 are a ribonucleotide,
TCAGAGCATGGAAATAAAACTT (SEQ ID NO: 18), wherein at least 3 consecutive nucleotides at positions 10-14 are a ribonucleotide.

25. The kit according to claim 24, wherein the third oligonucleotide is one or more selected from the group consisting of the oligonucleotides of SEQ ID NOs: 12-14:
ATAGGCTTrGrArArGCAGTGCA (SEQ ID NO: 12), wherein rG and rA at positions 9-12 are each ribonucleotides,
ATAGGCTTrGrArArGCAGTGCAT (SEQ ID NO: 13), wherein rG and rA at positions 9-12 are each ribonucleotides, and
TCAGAGCATGrGrArArATAAAACTT (SEQ ID NO: 14), wherein rG and rA at positions 11-14 are each ribonucleotides.

26. The method according to claim 23, wherein the detectable label is a fluorescence resonance energy transfer pair.

27. The method according to claim 23, wherein the amplifying is conducted using a method selected from the group consisting of Polymerase Chain Reaction, Ligase Chain Reaction, Self-Sustained Sequence Replication, Strand Displacement Amplification, Transcriptional Amplification System, Q-Beta Replicase, Nucleic Acid Sequence Based Amplification, Cleavage Fragment Length Polymorphism, Isothermal and Chimeric Primer-initiated Amplification of Nucleic Acid, and Ramification-extension Amplification Method.

28. The method according to claim 23, wherein the amplifying, the hybridizing and the contacting are simultaneously or sequentially carried out.

29. The kit according to claim 8, further comprising
(d) an amplifying activity for a PCR amplification of the target DNA sequence to produce a E. coli O157:H7 PCR fragment;
(e) an RNase H activity;
(f) a reverse transcriptase activity for reverse transcription of the E. coli O157:H7.

30. The kit according to claim 29, further comprising positive, internal, and negative controls.

31. The kit according to claim 30, further comprising uracil-N-glycosylase.

32. The kit according to claim 29, wherein the detectable marker is a fluorescent label.

33. The kit according to claim 32, wherein the probe is labeled with a FRET pair.

34. The kit according to claim 29, wherein the probe is immobilized to a solid support.

35. The kit according to claim 29, wherein the probe is in free form in a solution.

36. The kit according to claim 29, which further comprises an amplification buffer.

37. The kit according to claim 29, which further comprises an amplifying polymerase activity.

38. The kit according to claim 29, wherein the RNase H activity is the activity of a thermostable RNase H.

39. The kit according to claim 29, wherein the RNase H activity is a hot start RNase H activity.

40. The kit according to claim 29, wherein the probe comprises the sequence of SEQ ID NO: 17 or SEQ ID NO: 18:
ATAGGCTTGAAGCAGTGCAX₁ (SEQ ID NO: 17), wherein X₁ is absence or T, and at least 3 consecutive nucleotides at positions 9-14 are a ribonucleotide,
TCAGAGCATGGAAATAAAACTT (SEQ ID NO: 18), wherein at least 3 consecutive nucleotides at positions 10-14 are a ribonucleotide.

41. The kit according to claim 29, wherein the third oligonucleotide is one or more selected from the group consisting of the oligonucleotides of SEQ ID NOs: 12-14:
ATAGGCTTrGrArArGCAGTGCA (SEQ ID NO: 12), wherein rG and rA at positions 9-12 are each ribonucleotides,
ATAGGCTTrGrArArGCAGTGCAT (SEQ ID NO: 13), wherein rG and rA at positions 9-12 are each ribonucleotides, and
TCAGAGCATGrGrArArATAAAACTT (SEQ ID NO: 14), wherein rG and rA at positions 11-14 are each ribonucleotides.

42. A method of detecting E. coli O157:H7 in a sample, the method comprising:
(a) reverse transcribing the E. coli O157:H7 target RNA in the presence of a reverse transcriptase activity and the reverse amplification primer to produce a target cDNA of the target RNA;
(b) amplifying the target cDNA sequence to produce an increased number of copies of the target nucleic acid, the amplifying including hybridizing a first primer comprising at least 10 consecutive nucleotides of the sequence of SEQ ID NO: 16, 3, 4, or 6 and a second primer comprising at least consecutive nucleotides of the sequence of SEQ ID NO: 7, 8, 9, 10, or 11 to the target cDNA to obtain a hybridized product of the target nucleic acid and the primers, and extending the first and the second primers of the hybridized product using a template-dependent nucleic acid polymerase to produce an extended primer product;
(c) hybridizing the target nucleic acid to at least one probe oligonucleotide which is substantially complimentary to the target cDNA to obtain a hybridized product of the target nucleic acid:probe oligonucleotide, wherein the probe comprises a DNA sequence and an RNA sequence and is coupled to a detectable label;
(d) contacting the hybridized product of the target nucleic acid:probe oligonucleotide to an RNase H to cleave the probes; and
(e) detecting an increase in the emission of a signal from the label on the probe, wherein the increase in signal indicates the presence of the E. Coli O157:H7 target RNA in the sample.

42. The method according to claim 41, wherein the probe oligonucleotide is the oligonucleotide of SEQ ID NO: 6 or 8:
TGAGACCGTGTCTrGTTACATTCG (SEQ ID NO: 6), wherein the nucleotide “rG” at position 14 is a ribonucleotide, and
CGAATGTAACAGACACGGTCTCA (SEQ ID NO: 8), wherein at least one of the nucleotides at positions 9, 10, 11, 12, and 13 is a ribonucleotide.

43. The method according to claim 42, wherein the probe oligonucleotide comprises the sequence selected from the group consisting of the oligonucleotides of SEQ ID NOs: 17 or 18:
ATAGGCTTGAAGCAGTGCAXᵢ (SEQ ID NO: 17), wherein X₁ is absence or T, and at least 3 consecutive nucleotides at positions 9-14 are a ribonucleotide,
TCAGAGCATGGAAATAAAACTT (SEQ ID NO: 18), wherein at least 3 consecutive nucleotides at positions 10-14 are a ribonucleotide.

44. The kit according to claim 43, wherein the third oligonucleotide is one or more selected from the group consisting of the oligonucleotides of SEQ ID NOs: 12-14:
ATAGGCTTrGrArArGCAGTGCA (SEQ ID NO: 12), wherein rG and rA at positions 9-12 are each ribonucleotides,
ATAGGCTTrGrArArGCAGTGCAT (SEQ ID NO: 13), wherein rG and rA at positions 9-12 are each ribonucleotides, and
TCAGAGCATGrGrArArATAAAACTT (SEQ ID NO: 14), wherein rG and rA at positions 11-14 are each ribonucleotides.

45. The method according to claim 42, wherein the detectable label is a fluorescence resonance energy transfer pair.

46. The method according to claim 42, wherein the amplifying is conducted using a method selected from the group consisting of Polymerase Chain Reaction, Ligase Chain Reaction, Self-Sustained Sequence Replication, Strand Displacement Amplification, Transcriptional Amplification System, Q-Beta Replicase, Nucleic Acid Sequence Based Amplification, Cleavage Fragment Length Polymorphism, Isothermal and Chimeric Primer-initiated Amplification of Nucleic Acid, and Ramification-extension Amplification Method.

46. The method according to claim 42, wherein the amplifying, the hybridizing and the contacting are simultaneously or sequentially carried out.

47. The kit according to claim 8, which comprises the first oligonucleotide of SEQ ID NO: 2, the second oligonucleotide of SEQ ID NO: 7, and the third oligonucleotide of SEQ ID NO: 13.

48. The kit according to claim 29, which comprises the first oligonucleotide of SEQ ID NO: 2, the second oligonucleotide of SEQ ID NO: 7, and the third oligonucleotide of SEQ ID NO: 13.
